(19) United States
(12) Patent Application Publication
(10) Pub. No.: US 2005/0244499 A1
(43) Pub. Date: Nov. 3, 2005

(54) Method and device for reducing susceptibility to fractures in long bones

(76) Inventors: Robert Diaz, Palm Beach Gardens, FL (US); David R. Campbell, Jupiter, FL (US)

Correspondence Address: MCHALE & SLAVIN, P.A., 2855 PGA BLVD, PALM BEACH GARDENS, FL, 33410, US

(21) Appl. No.: 10/838,523

(22) Filed: May 3, 2004

(51) Int. Cl. A61K 9/14; A61M 31/00

(52) U.S. Cl. 424/484; 604/500

(57) ABSTRACT

The invention provides a method and a device for administering bone matrix with or without additional bone growth enhancing agents, or administering one or more bone growth enhancing agents to the interior surface of a long bone and to the cancellous bone within the medullary cavity to enhance bone growth and strength, thus reducing susceptibility of the bone to fracture.

### FIELD OF THE INVENTION

The instant invention relates generally to methods useful for the prevention of fractures in bones; particularly to the prevention of fractures in bones which are at increased risk for fracture with minimum trauma and most particularly to administration of bone matrix with or without additional bone growth enhancing agents, or to administration of one or more bone growth enhancing agents to the interior surface of a long bone to enhance bone growth and strength, thus reducing susceptibility of the long bone to fracture.

### BACKGROUND OF THE INVENTION

Bones provide an organism support and protection, for example, support for muscle movement and protection for organs. Living bone tissue is in a constant state of flux due to the process of bone remodeling. In the process of bone remodeling, the mineralized bone matrix is continuously deposited and resorbed. Bone cells termed “osteoclasts” and “osteoblasts” carry out bone remodeling. Osteoclasts remove tissue from the bone surface and osteoblasts replace this tissue.

Rapid turnover of bone occurs throughout childhood as bones increase in size and thickness until the individual reaches a genetically-determined adult height. At adult height bones cease to grow in size but continue to increase in thickness until the individual reaches approximately 30 years of age. As bone growth ceases, the activity of osteoblasts and osteoclasts becomes imbalanced and bone is resorbed faster than it is replaced, thus leading to a gradual thinning of the bones. With thinning the microarchitexture of bone tissue deteriorates creating spaces or pores between the normally dense units of the bony matrix.

“Porous bone”, a pathological condition termed “osteoporosis” occurs with chronic thinning of bones. The hallmark of osteoporosis is increased fragility of bones due to the loss of bone from the interior of the medullary canal. Such bone loss reduces the overall density of bone tissue (osteopenia). As a bone thins it becomes increasingly susceptible to fracture with minimum trauma. Ideally, therapeutic measures for thinning bone should restore bone density and thus reduce susceptibility to fracture. Preventing fracture of osteoporotic bone, significantly improves the health, well-being and functional capabilities of the osteoporotic patient.

Other bone-related diseases and/or defects may involve thinning of the bones, for example, after a traumatic injury to a limb with resultant osteopenia, corticosteroid regimens, complications with prosthetic devices and damage due to radiation treatments.

Although there is much information in the art regarding factors and methods which can influence bone remodeling, information is more limited on factors and methods which can directly stimulate bone growth in general. What is needed in the art is an efficient method which can achieve enhanced bone growth in areas specifically affected by osteopenia, thus increasing bone density in these affected areas and reducing susceptibility of the thinning bones to fracture.

### DESCRIPTION OF THE PRIOR ART

Numerous and varied treatments for osteoporosis can be found in the prior art; a few examples of such treatments follow.

U.S. Pat. Nos. 4,904,478 and 5,228,445 disclose the use of a slow release sodium fluoride preparation which when administered maintains a safe and effective serum level of fluoride useful for the treatment of osteoporosis. This preparation stimulates bone formation and improves bone quality thus aiding in the prevention of bone fractures which are often a frequent occurrence in osteoporetic patients.

U.S. Pat. No. 5,614,496 discloses a method for administration of FGF-1 in order to promote bone repair and growth.

U.S. Pat. No. 5,663,195 discloses a method of inhibiting bone resorption by administration of a selective cyclooxygenase-2 inhibitor. This method halts or retards loss of bone, promotes bone repair and aids in prevention of fractures.

U.S. Pat. Nos. 5,763,416 and 5,942,496 disclose methods for the transfer of osteotropic genes (genes for parathyroid hormone, BMP's, growth factors, growth factor receptors, cytokines and chemotactic factors) into bone cells for treatment of bone-related diseases and defects.

U.S. Pat. No. 5,962,427 discloses a method for specific targeting and DNA transfer of a therapeutic gene into mammalian repair cells. The modified repair cells proliferate and populate a wound site while expressing the therapeutic gene.

Dr. Brunilda Nazario reports on a drug, FORTEO (teriparatide), derived from parathyroid hormone, which is useful in the treatment of osteoporosis (accessed from the WebMD website on Dec. 23, 2003). Teriparatide is a bone formation agent that promotes bone growth by increasing the number and activity of bone-forming cells (osteoblasts).

A substantial amount of research has been conducted to elucidate methods for improved healing of skeletal defects; resulting in, for example, immobilization devices and bone grafts.

Many devices have been constructed for application to the area of a bone fracture in order to immobilize, facilitate and support healing and prevent deformities, such as the devices disclosed in U.S. Pat. No. 5,853,380; U.S. Pat. No. 5,941,877; U.S. application 2003;0181979 and U.S. application 2003; 0099630. Methods involving the replacement of damaged bone tissue with a bone graft are more common. A bone graft can be prepared from autograft tissue (bone tissue is obtained from a site other than the damaged bone area in the same individual requiring the graft), allograft tissue (bone tissue is obtained from a donor) or can be constructed from artificial materials.

Use of allograft tissue avoids donor site complications in the tissue recipient, additionally such tissue can be obtained in large quantities. However, many disadvantages arise when using allograft tissue, including, expense, possible disease transmission and detrimental host response. Allan E. Gross (Orthopedics 26(9):927-928 September 2003) discusses use of allograft tissue in reconstructive surgery in the lower extremities.

Currently, autograft remains the treatment of choice, however due to the increased need for bone tissue occurring during the past decade other materials have been developed as a substitute for or as a means to extend a bone graft.

Victor Goldberg (Orthopedics 26(9):923-924 September 2003) presents a general discussion of the biology of bone grafts. Such knowledge aids in selection of the appropriate graft for each clinical application, since no single material is suitable for every purpose.

Bauer et al. (Orthopedics 26(9):925-926 September 2003) present a general discussion of four categories of available bone graft substitutes; hydroxyapatite products, soluble calcium-based blocks/granules, injectable cements and osteoinductive materials.

Generally derived from sea coral, hydroxyapatite products are osteoinductive and possess compressive strength. These products can be brittle, difficult to prepare and slow to resorb once implanted. Examples of the use of hydroxyapatite products in bone tissue repair can be found, for example, in U.S. Pat. Nos. 6,585,992; 6,290,982; 6,206,957; 5,069,905 and 5,015,677.

Soluble calcium-based blocks/granules facilitate the mineral deposition which is necessary for bone remodeling. Lee Beadling (Orthopedics Today, page 43, November 2003) discloses an injectable calcium sulfate graft having improved compressive strength and resorption properties.

Yu et al. (U.S. application 2002;0169210, published on Nov. 14, 2002) disclose a method for treating and preventing fractures with administration of calcium L-threonate. Calcium L-threonate was found to promote proliferation, differentiation and mineralization of osteoblasts and also found to promote expression of collagenI mRNA in osteoblasts. Yu et al. disclose that treatment with calcium L-threonate facilitated bone fracture healing and increased bone density and mechanical performance thus preventing bone fracture. In the method of Yu et al. calcium L-threonate was taken systemically (orally or parentally) and was not applied directly to the desired location in specific bones as in the method of the instant invention.

Cements which are capable of injection at fracture sites or sites of implantation of prosthetic devices act as bonding material for improving fracture healing and for securing prosthetic devices. Injectable cements vary in useful properties; for example; calcium phosphate is osteoconductive, has compressive strength, slow resorption, and is weak in tensile strength and shear while silica based cements are strong but weakly osteoinductive. There are many cements and devices for their use known in the art, for example, the isovolumic mixing and injection device disclosed by James Marino in U.S. Pat. No. 6,406,175.

Demineralized human bone tissue, termed bone matrix when mixed with a carrier such as glycerol, is powerfully osteoinductive and naturally contains growth factors which aid in healing bone, such as bone morphogenetic proteins (BMP's). BMP's were first identified from demineralized bone and were found to function as signal transducing proteins in the processes of skeletal development and bone formation. Currently, BMP's are under clinical investigation as potential facilitators of bone and cartilage repair.

Cheng et al. (The Journal of Bone and Joint Surgery 85-A (8):1544-1552 2003) present a review of the osteogenic functions attributed to fourteen types of BMP's.

Issack et al. (The American Journal of Orthopedics pages 429-436 September 2003) present a review discussing advances toward clinical application of BMP's in bone and cartilage repair. Issack et al. note animal studies which demonstrated the osteogenic and chondrogenic potential of BMP's and additionally note human clinical trials which demonstrated the ability of BMP's to enhance spinal fusion, promote union of fractured bones and heal size defects.

Thomas A. Einhorn (The Journal of Bone and Joint Surgery 85-A (Supplement 3):82-88 2003) also presents a review discussing clinical applications of recombinant human BMP's. Einhorn notes clinical trials which demonstrated the ability of BMP's to enhance the healing of fractures and spinal defects and to enhance spine and joint arthrodeses.

Sandhu et al. (The Journal of Bone and Joint Surgery 85-A (Supplement 3):89-95 2003) disclose a study that demonstrated successful use of BMP-2 to enhance spinal fusion.

Einhorn et al. (The Journal of Bone and Joint Surgery 85-A (8):1425-1435 2003) disclose a study wherein a single, local, percutaneous injection of rhBMP-2 was shown to accelerate fracture-healing in a rat femoral fracture model.

In contrast to the instant invention, the prior art does not disclose the use of BMP's for prevention of fractures in an unfractured bone or in a bone susceptible to fracture before fracture occurs. The instant inventors are the first to contemplate administration of BMP's to unfractured bone for the prevention of fractures.

### SUMMARY OF THE INVENTION

The instant invention provides an efficient method for achieving enhanced bone growth in areas specifically affected by osteopenia, thus increasing bone density in these affected areas and reducing susceptibility of the thinning bones to fracture. The method is particularly suited to the treatment of long bones and generally is accomplished through carrying out three basic steps; formulating a bone matrix/bone growth enhancing solution, administering said solution to an interior surface of a long bone and distributing said solution along the interior surface longitudinal length of a long bone. The method may be practiced separately or practiced in consort with other procedures, for example (but not limited to), with joint replacement surgery and with surgical repair of fractures.

The first step involves formulating a solution including bone matrix and/or at least one bone growth enhancing agent. A solution may include bone matrix alone, a bone growth enhancing agent alone or combinations of bone matrix and bone growth enhancing agents. Bone matrix may be combined with a single bone growth enhancing agent or with multiple bone growth enhancing agents. Any material which enhances bone growth is contemplated for use in the solution of the instant invention; illustrative, albeit non-limiting examples of such materials are bone morphogenetic proteins (BMP's), cytokines, hormones and growth factors.

The instant invention also provides means for administration of the solution. The means for administration is a device constructed and arranged for controlled deposition of the solution into the medullary cavity and onto the interior surface of the bone. The form of the device may be illustrated as a standard cannula shaft having at least one insert. Since the rate of bone thinning varies for each individual and even varies at different rates in separate areas of the same individual, one insert design may not be ideally suited to every situation. One of skill in the art would have the knowledge to choose the design best suited for each individual situation.

The second step of the method involves administration of the solution to an interior surface of a long bone having a predetermined longitudinal length by use of a device inserted into it's intramedullary canal through an aperture formed along a longitudinal end of a long bone.

The third step of the method involves distribution of the solution along substantially the entire longitudinal length of the interior surface of a long bone in a way that allows the solution contact with the cortical tissue effective for achieving active bone restoration as a result of controlled deposition of the solution. Additionally, the solution will disperse, by flowing through the cancellous bone channels, to contact the cancellous and trabecular bone of the shaft of a long bone.

Additionally, the step of distributing may further include mechanical disruption of the interior surface of the bone. The mechanical disruption must be sufficient to cause an inflammatory response since such a response improves the efficacy of the method by promoting uptake of the solution by the bone tissue. The tip of the cannula may thus be designed to carry out mechanical disruption of the interior surface of the bone and such disruption may occur prior to, subsequent to or simultaneously with the distribution of the solution. The solution may be administered in a single dose, in multiple doses over periods of time or formulated for controlled release after administration, e.g. formulated within a carrier of limited solubility, encapsulated within a slowly degrading matrix, or the like.

Although the method and device of the instant invention are exemplified by administration to an unfractured bone which has been determined to be at risk for fracture (at-risk bone), they may also be administered to a fractured bone to improve healing by enhancing growth of the newly formed bone. The instant invention is contemplated for use with any bone-related disease and/or defect which may involve thinning, weakened and/or damaged bones; illustrative, albeit non-limiting situations are, osteoporosis, after a traumatic injury to a limb with resultant osteopenia, corticosteroid regimens, osteogenesis imperfecta, complications with prosthetic devices and bone damage due to radiation treatments.

Accordingly, it is an objective of the instant invention to provide a method for reducing susceptibility to fractures in bones comprising administration of a solution including bone matrix and/or at least one bone growth enhancing agent to an interior surface of a bone.

It is another objective of the instant invention to provide a method for reducing susceptibility to fractures in bones comprising administration of a solution including bone matrix and/or at least one bone growth enhancing agent to an interior surface of a bone wherein said step of administering further includes mechanical disruption of an interior surface of a bone sufficient to induce an inflammatory response.

It is yet another objective of the instant invention to provide a device constructed and arranged for controlled deposition of a solution into the medullary cavity and onto the interior surface of a bone.

Other objectives and advantages of the instant invention will become apparent from the following description taken in conjunction with the accompanying drawing(s) wherein are set forth, by way of illustration and example, certain embodiments of the instant invention. The drawing(s) constitute a part of this specification and include exemplary embodiments of the present invention and illustrate various objects and features thereof.

### BRIEF DESCRIPTION OF THE FIGURES

FIG. 1 illustrates the first general step in one embodiment of the invention.

FIG. 2 illustrates the second and third general steps in one embodiment of the invention.

### ABBREVIATIONS AND DEFINITIONS

The following list defines terms, phrases and abbreviations used throughout the instant specification. Although the terms, phrases and abbreviations are listed in the singular tense the definitions are intended to encompass all grammatical forms.

As used herein, the term “osteoplasty” refers to any surgical procedure or process by which total or partial loss of bone is remedied.

As used herein, the term “bone mineral density test” refers an X-ray process wherein the amount of calcium in bones is determined and bone strength is ascertained. The most common areas for application of bone mineral density testing are the hip and spine. This test is used most often to detect osteoporosis.

As used herein, the abbreviation “DEXA” refers to dual energy X-ray absorptiometry; a type of bone mineral density testing wherein two X-ray beams are applied to the bone and the amounts of each X-ray beam blocked by bone and tissue are compared to estimate bone density.

As used herein, the abbreviation “P-DEXA” refers to a modification of the DEXA test wherein bone density in peripheral bone areas such as the wrist is measured.

As used herein, the abbreviation “DPA” refers to dual photon absorptiometry; a type of bone mineral density test similar in principle to the DEXA test; but instead uses a radioactive material to produce photons which are applied to bone (in place of X-ray beams).

As used herein, the term “ultrasound” refers to a type of bone mineral density test which utilizes sound waves reflected from bones in peripheral areas of the body to measure bone density.

As used herein, the term “cannula” refers to a tube for insertion into a body cavity, duct or vessel generally functioning as a vehicle for introduction of the inserts of the device of the instant invention. A cannula can be modified according to body area of and type of delivery desired. The cannula of the instant invention has at least one insert.

As used herein, the term “device” refers to a standard cannula shaft having one or more inserts. The term “device” is used interchangeably with the term “means”.

As used herein, the phrase “at-risk bone” refers to a bone which has been determined to be at risk for fracture; due to identified fragility, presence adjacent to a fractured bone or other identifiable risk factors for fracture known to those of skill in the art.

As used herein, the term “bone matrix” refers to human bone tissue which has been demineralized and combined with a carrier material such as glycerol or starch. Bone matrix naturally contains bone growth enhancing agents.

As used herein, the term “bone growth enhancing agent” refers to any injectable biological and/or synthetic molecule or material which facilitates and/or increases the rate of bone growth and is capable of combination with bone matrix. A bone growth enhancing agent can also be referred to as a bone growth accelerator.

As used herein, the phrase “at least a substantial length” refers to the amount of longitudinal interior surface area of cortical bone covered as a result of administration of the solution. The amount of longitudinal interior surface area covered must be an amount effective to achieve active bone restoration.

As used herein, the term “controlled deposition” refers to the ability of the device for distribution of the bone matrix solution to control internal pressure of solution release, to control amount of solution released and to control mechanical disruption of the interior surface area of the bone. The viscosity of the solution is also controlled to assure a precise location of the solution in the intramedullary canal and to prevent extrusion into the extraosseus space.

As used herein, the abbreviation “BMP” refers to bone morphogenetic protein. “rhBMP” refers to recombinant, human bone morphogenetic protein. BMP's are signal transducting proteins of the transforming growth factor-beta superfamily which function in skeletal development and bone formation. BMP's were first identified in demineralized bone.

As used herein, the phrase “naturally contains” refers to any substance or material which occurs in nature or is naturally present in a living or previously living organism, for example, bone matrix as obtained from a human tissue donor naturally contains BMP's but does not naturally contain recombinant BMP's or other such recombinant proteins.

The terms “surgical wound” and “incision” are used interchangeably herein.

### DETAILED DESCRIPTION OF THE INVENTION

Thinning of bones occurs frequently with many bone diseases and/or defects. Thin bones are at an increased risk for fracture with minimum trauma. Many deleterious effects accompany bone fracture, such as, pain, immobility, deformity, increases in length and cost of healthcare, and a general reduction in the quality of life of the individual suffering the fracture. Bone fractures may even give rise to complications which may result in serious illness and death. The instant invention can circumvent these deleterious effects by providing a method for achieving active restoration of thinning bones. Such restoration increases bone density and thus increases bone strength leading to a reduction in susceptibility of the bone to fracture.

The method of the instant invention is particularly suited to the treatment of long bones and generally is accomplished through carrying out three basic steps; formulating a bone matrix/bone growth enhancing solution, administering said solution into the medullary cavity and onto the interior surface of a long bone and distributing said solution along the interior surface longitudinal length of a long bone.

The solution, as formulated according to the instant invention, may include bone matrix alone, a bone growth enhancing agent alone or combinations of bone matrix and bone growth enhancing agents. Any bone cement known in the art can also be added to the solution or can replace the bone matrix in the solution. Bone matrix may be combined with a single bone growth enhancing agent or with multiple bone growth enhancing agents. As bone matrix is derived from human bone tissue, it naturally contains bone growth enhancing agents. The addition of at least one bone growth enhancing agent to the bone matrix solution may increase the effectiveness of the treatment. Additional bone growth enhancing agents can be obtained from any tissue source or can be recombinantly produced. Any natural and/or synthetic material which enhances bone growth is contemplated for use in the solution of the instant invention, illustrative, albeit non-limiting examples of such materials are BMP's, cytokines, hormones and growth factors. Illustrative, albeit non-limiting examples of BMP's are any of the fourteen types of human BMP's (BMP's 1-14). Cytokines are polypeptides transiently produced by many different types of cells and function as intercellular messengers, usually by binding to cell surface receptors. Illustrative, albeit non-limiting examples of cytokines are interferons, tumor necrosis factors, lymphokines, colony-stimulating factors and erythropoietin. Hormones are also organic intercellular messengers. Illustrative, albeit non-limiting examples of hormones are steroid hormones, prostaglandins, peptide H, adrenalin and thyroxin. Growth factors are mitogenic polypeptides functioning in intercellular signaling. Illustrative, albeit non-limiting examples of growth factors are platelet derived growth factor, transforming growth factors and epidermal growth factor. The volume and concentration of solution will be formulated on a per case basis since volume and concentration of the solution depends on the length and volume of the bone to be treated. The quality (degree of thinning) of the bone to be treated determines the type of administration, for example, a single dose of solution, multiple doses of solution over a period of time, or a solution formulated for controlled release after administration. A radioopaque material can also be added (to the solution) in order to facilitate visualization of the administration and distribution of the solution.

Additionally, the instant invention provides means for administration of the solution. The means for administration is a device constructed and arranged for controlled deposition of the solution into the medullary cavity and onto the interior surface of the bone. The form of the device may be illustrated as a standard cannula shaft having at least one insert. Since the rate of bone thinning varies for each individual and even varies at different rates in separate areas of the same individual, one insert design may not be ideally suited to every situation. One of skill in the art would have the knowledge to choose the design best suited for each individual situation.

After preparation of the solution and the device, an incision is made in the tissue (including the bone) in order to form an intramedullary aperture for insertion of the device. The incision must be of a width sufficient for insertion and maneuverability of the device within the interior cavity of the long bone. Bi-planar fluoroscopic or image-guided systems are used to guide the introduction of the device into the bone.

The second step of the method involves administration of the solution to an interior surface of a long bone by use of a device constructed according to the predetermined longitudinal length of the long bone to be treated. The device is inserted through the aperture created by the incision and positioned in a manner such that the device is parallel to the longitudinal axis of the long bone.

After insertion of the cannula, the solution is distributed (third step of the general method) along substantially the entire longitudinal length of the interior surface of a long bone and diffuses into the medullary cavity in a way that allows the solution contact with the cortical and cancellous tissue effective for achieving active bone restoration. Distribution may be carried out by spraying or injecting the solution. Additionally, the step of distributing may further include mechanical disruption of the interior surface of the bone. Mechanical disruption may be carried out by scraping, tapping and/or applying pressure to the interior surface of the long bone. The tip of an insert may thus be designed to carry out mechanical disruption of the interior surface of the bone and such disruption may occur prior to, subsequent to or simultaneously with the distribution of the solution. One embodiment of the device is composed of a standard cannula shaft comprising a tube constructed to deliver the solution and at least two additional tubes. Each of the additional tubes has blades at the end which when advanced through the end of the cannula shaft spring out and expand to contact the bone and to distribute the solution along the entire interior cavity. The mechanical disruption must be sufficient to cause an inflammatory response in the disrupted bone tissue since such a response improves the efficacy of the method by promoting uptake of the solution by the bone tissue. The distribution of solution should always be carried out by “controlled deposition”. Controlling the deposition of the solution is necessary to assure that precise amounts of solution are distributed in a manner which avoids unintentional fracture, excessive mechanical disruption or extrusion of the solution into extraosseus locations.

After the distribution, the device is withdrawn, a means for preventing extrusion from the entry point is applied, e.g. a cement blocker, plug or the like is inserted into entry site of the device to prevent extrusion and a suture is prepared to close the incision.

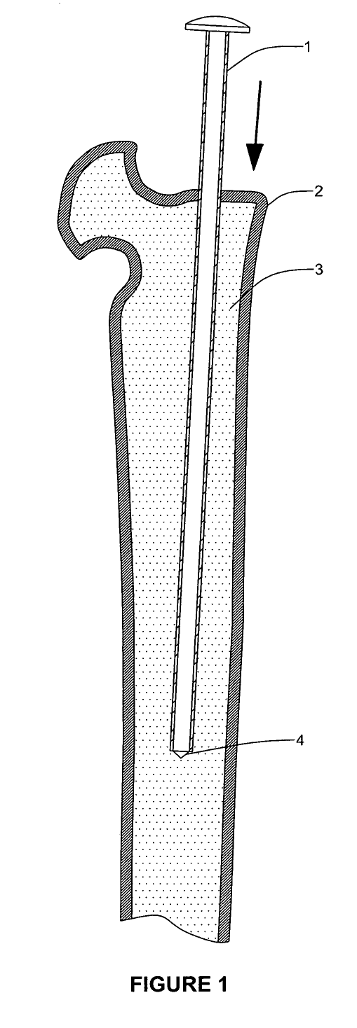

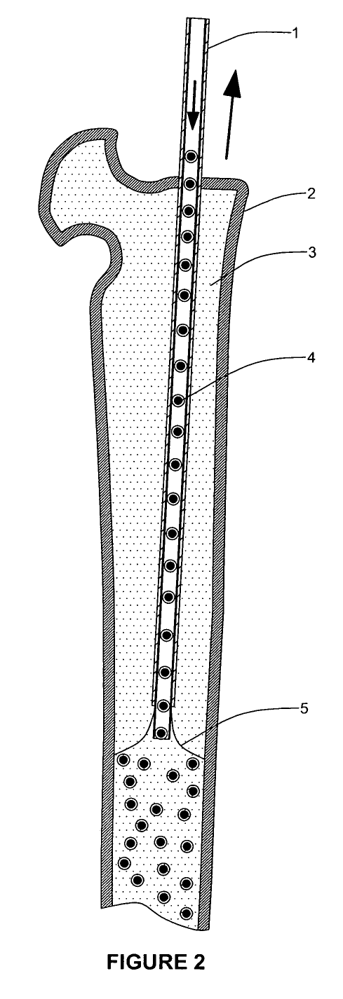

FIGS. 1 and 2 show one embodiment of the instant invention. FIG. 1 illustrates the introduction of the device into the medullary cavity of a non-fractured femur. The cannula shaft is not illustrated. Insert 1 is guided through the periosteum 2 along the longitudinal length of the medullary cavity (direction shown by black arrow). Blade 4 provides for mechanical disruption of the cortex 3. FIG. 2 illustrates delivery of the solution into the medullary cavity of the non-fractured femur after insert 1 of FIG. 1 is withdrawn through the periosteum 2 along the cortex 3. The cannula shaft is not illustrated. A fenestrated mesh bag 5 is delivered into the medullary cavity through insert 1 (FIG. 2). Pellets 4 are delivered through insert 1 filling the fenestrated mesh bag. Pellets 4 comprise ceramic beads having bound recombinant human BMP. As pellets 4 are filling the fenestrated mesh bag 5, air, bone marrow and other interior bone materials are displaced and extruded (direction shown by black arrows). Enclosing pellets 4 in a fenestrated mesh bag 5 provides interior support to the femur and keeps the pellets 4 in place thus limiting extrusion of the pellets Other growth factors and/or bone growth accelerators may be added to the bag via additional inserts and/or cannulas if desired.

Alternative embodiments would utilize multiple disruption blades (not shown), in place of the single blade 4 as illustrated above, and would utilize bone growth materials in alternative vehicles or carriers in the form of a solution, suspension, controlled release formulation or the like.

Another embodiment of the method is illustrated by example.

### EXAMPLE

The following protocol is designed to be carried out to treat an individual with a thinning femur and would be implemented either as a separate surgical procedure or in conjunction with another surgical procedure, such as a hip or knee joint replacement.
1. One would measure the length of the femur to be treated in order to determine the amount of bone matrix solution required and the length of device required for the treatment of the particular individual;
2. One would X-ray the femur to be treated and/or perform bone density tests in order to determine bone quality (degree of thinning) for selection of the type of formulation of the bone matrix solution required for the treatment of the particular individual;
3. One would then prepare the bone matrix solution in the predetermined amount and formulation, adding additional bone growth enhancing agents if desired;
4. One would then select the desired insert design of predetermined length and load the selected insert with the formulated bone matrix solution and load the insert into the cannula shaft;
5. One would then either prepare an incision in the tissue (including the bone) or utilize an incision prepared for an additional surgical procedure which is of significant width to allow insertion and maneuverability of the device in the medullary cavity of the femur to be treated;
6. One would then insert the device through the aperture in the skin, soft tissue and bone created by the incision, position the device within the medullary canal such that the end of the insert is positioned in the area to be treated and engage the device to administer the bone matrix solution by either injection or spray;
7. One would then distribute the bone matrix solution by controlled deposition along the area to be treated in a way that allows the bone matrix solution contact with the cortical and cancellous tissues effective for achieving active bone restoration;
8. One would then engage the device for withdrawal through the medullary canal and then optionally engage the cortical surface of the femur with the tip of the insert while withdrawing in order to mechanically disrupt the cortical surface sufficient to cause an inflammatory response, as such a response promotes uptake of the solution by the bone tissue;
9. After withdrawal of the device, one would insert a cement blocker or plug at the entry site of the device to prevent leakage of the solution from the medullary canal. After insertion of the cement blocker or plug, one would prepare a suture to close the incision and complete the procedure.

The post-procedure follow-up of the individual would include X-Rays and/or several bone density tests over a period of time in order to track the bone restoration in the treated femur.

As evidenced by the above discussion and illustrated by the figures and the example, the method of the instant invention achieves active bone restoration and thus decreases the susceptibility of thinning bones to fracture. Practice of the above invention may improve treatment of bone diseases and/or defects having resultant osteopenia including osteoporosis, after a traumatic injury to a limb, corticosteroid regimens, complications with prosthetic devices and damage due to radiation treatments.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The various bone matrices, bone growth enhancing compounds, bone cements, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method for reducing susceptibility to fracture in a long bone comprising the steps of:
(a) formulating a bone matrix solution;
(b) administering said solution to an interior surface of a long bone having a predetermined longitudinal length by use of a device inserted through an aperture into the intramedullary canal formed along a longitudinal end of said long bone; and
(c) distributing said solution along substantially said entire longitudinal length of said interior surface of said long bone whereby at least a substantial length of said interior surface is coated with said solution, wherein enhanced bone growth is achieved thereby reducing susceptibility of said long bone to fracture.

2. The method in accordance with claim 1 wherein said bone matrix solution of step (a) further includes at least one additional bone growth enhancing agent.

3. The method in accordance with claim 2 wherein said at least one additional bone growth enhancing agent is selected from the group consisting of bone morphogenetic proteins (BMP's), cytokines, hormones, growth factors and combinations thereof.

4. The method as in any one preceding claim, in which said long bone is unfractured.

5. The method as in any one preceding claim, in which said step of distributing comprises spraying or injecting said bone matrix solution of step (a).

6. The method in accordance with claim 5 wherein said step of distributing further includes mechanical disruption of said interior surface of said long bone sufficient to induce an inflammatory response.

7. The method in accordance with claim 6 wherein said mechanical disruption occurs prior to said step of administering.

8. The method in accordance with claim 6 wherein said mechanical disruption occurs subsequent to said step of administering.

9. The method in accordance with claim 6 wherein said mechanical disruption occurs simultaneously with said step of administering.

10. The method as in any one preceding claim, in which said bone matrix solution of step (a) is further formulated for controlled release of said solution.

11. The method as in any one preceding claim, in which said device of step (b) is constructed and arranged for controlled deposition of said bone matrix solution upon said interior surface of said long bone.

12. A method for reducing susceptibility to fracture in a long bone comprising the steps of:
(a) formulating a solution including a bone matrix and at least one bone growth enhancing agent wherein said at least one bone growth enhancing agent is selected from the group consisting of bone morphogenetic proteins (BMP's), cytokines, hormones, growth factors and combinations thereof;
(b) providing means for administering the solution of step (a) to an interior surface of said long bone wherein said means are constructed in a length essentially equivalent to a length of said long bone to which said solution is administered and wherein said means are constructed and arranged for controlled deposition of said solution upon said interior surface of said long bone;
c) preparing an incision within a tissue surrounding said long bone in a manner that allows access to an interior of said long bone to which said solution is administered wherein said incision is of a width sufficient for maneuverability of said means within said interior of said long bone;
(d) inserting said means through said incision to access said interior of said long bone to which said solution is administered wherein said inserting is parallel to a longitudinal axis of said long bone;
(e) administering said solution in a manner such that said interior surface of said long bone is at least partially coated with said solution;
(f) withdrawing said means for administering parallel to said longitudinal axis of said long bone through said incision;
(g) inserting a means for preventing extrusion within said entry site of said means for administering into said tissue; and
(h) preparing a suture to close said incision, whereupon closure said administration achieves enhanced bone growth thereby reducing susceptibility of said long bone to fracture.

13. The method in accordance with claim 12 wherein said long bone is unfractured.

14. The method in accordance with claim 12 wherein said step of administrating further includes mechanical disruption of said interior surface of said long bone sufficient to induce an inflammatory response.

15. The method in accordance with claim 14 wherein said mechanical disruption occurs prior to said step of administering.

16. The method in accordance with claim 14 wherein said mechanical disruption occurs subsequent to said step of administering.

17. The method in accordance with claim 14 wherein said mechanical disruption occurs simultaneously with said step of administering.

18. The method as in any one of claims 12-17, in which said solution of step (a) is further formulated for controlled release of said solution.

19. The method as in any one of claims 12-17, in which said step of distributing comprises spraying or injecting said bone matrix solution of step (a).

20. The method as in claim 18 in which said step of distributing comprises spraying or injecting said bone matrix solution of step (a).
